(19) United States
(12) Patent Application Publication
(10) Pub. No.: US 2012/0059386 A1
(43) Pub. Date: Mar. 8, 2012

(54) Vacuum Controlled Capsulorhexis Device

(76) Inventor: Glenn Robert Sussman, Laguna Niguel, CA (US)

(21) Appl. No.: 12/877,456

(22) Filed: Sep. 8, 2010

(51) Int. Cl. A61F 9/00

(52) U.S. Cl. 606/107

(57) ABSTRACT

A capsulorhexis device is disclosed. The capsulorhexis device includes a tip portion, a vacuum channel, and a flexible membrane. The vacuum channel extends through the tip portion. Further, the vacuum channel is operatively connected to a vacuum source. The tip portion further includes a port formed therethrough. The port is in communication with the vacuum channel. The flexible membrane is positioned with the vacuum channel. The flexible membrane permits a predetermined level of vacuum to reach the port without causing aspiration of tissue through the vacuum channel.

### BACKGROUND

Cataracts are a common eye disease that involves the opacification of the material within the intraocular lens of the eye. Certain surgical techniques and instruments have been developed to extract the cataractous lens and replace the natural opacified intraocular lens with an artificial intraocular lens (IOL). In one part of cataract surgery, which is a multi-step surgical procedure, a capsulorhexis procedure may be performed. Basically, the capsulorhexis procedure begins by making a slit incision in the cornea. After the incision is made, the eye is entered with the cystotome to puncture the capsular bag. A small, circular incision is then made in the capsular bag. This circular incision provides an opening through which removal of the catearactous lens and insertion of the IOL is accomplished.

More specifically, capsulorhexis, also known as continuous curvilinear capsulorhexis, is a technique that defines a circular incision that is formed through continuous tearing of the anterior capsule bag. However, during phacoemulsification, there is a great deal of tension on the cut edges of the anterior capsulorhexis while the lens nucleus is emulsified. Accordingly, a continuous cut or tear (rhexis), without tags is a desired step in a safe and effective phacoemulsification procedure.

Indeed, if the capsule is opened with numerous small capsular tears, the small tags that remain can lead to radial capsular tears which may extend into the posterior capsule. Radial tears typically result in complications, as the tears destabilize the lens for further cataract removal and safe IOL placement within the lens capsule later in the operation. Further, if the posterior capsule is punctured during the capsulorhexis procedure, then the vitreous may gain access to the anterior chamber of the eye. In this situation, the vitreous may be removed by an additional procedure with special instruments. The loss of vitreous is also associated with an increased rate of subsequent retinal detachment and/or infection within the eye. These complications are potentially blinding.

Known prior art devices and methods used for the capsulorhexis procedure require a great deal of skill on the part of the surgeon to produce a continuous curvilinear capsular opening. This required skill is due to the difficulty in controlling the tearing action. More specifically, an initial capsular incision may be torn into a circular shape by grasping the leading edge (or flap) with fine caliber forceps and advancing the cut. The forceps grasp the flap and manually rotate it to form the circular incision. Often, the physician is required to continuously tear the tissue a predetermined length, then release and re-grasp the tissue flap. This approach involves a very challenging maneuver and the tearing motion can sometimes lead to an undesirable tear of the capsule toward the back of the lens, even in the most experienced hands. Further, opening and closing the forceps during this technique may cause trauma to the cornea. Trauma to the eye may also be caused by pressure exerted on the eye by the surgeon during the procedure.

Another potential issue with forceps is the size of initial slit incision made in the cornea. Because the forceps may need to open and close, the initial slit incision may need to have a minimum size to accommodate these actions. The larger the incision, the greater potential for tissue damage and trauma to the patient.

Moreover, because of the fine control required, and due to the precision needed, the current devices for capsulorhexis procedures may be made from high quality metal (titanium, for example). Brief Summary

A capsulorhexis device is disclosed. The capsulorhexis device includes a handle portion, a tip portion, a vacuum channel, and a flexible membrane. The vacuum channel extends through the tip portion and the handle portion. Further, the vacuum channel is operatively connected to a vacuum source. The tip portion further includes a port formed therethrough. The port is in communication with the vacuum channel. The flexible membrane is positioned with the vacuum channel. The flexible membrane permits a predetermined level of vacuum to reach the port without causing aspiration of tissue through the vacuum channel.

### BRIEF DESCRIPTION OF THE DRAWINGS

Embodiments of the present disclosure will now by described by way of example in greater detail with reference to the attached figures, in which:

FIG. 1 is a side, partial cross-sectional view of a distal end portion of a capsulorhexis device, prior to vacuum being applied;

FIG. 2 is a side, partial cross-sectional view of the distal end portion of the capsulorhexis device after vacuum has been applied;

FIG. 3a is a perspective view of an exemplary attachment arrangement for the capsulorhexis device in a disconnected configuration;

FIG. 3b is a perspective view of the attachment arrangement of the capsulorhexis device in a connected configuration; and

FIG. 4 is a block diagram of the capsulorhexis device and a vacuum source.

### DETAILED DESCRIPTION

Referring now to the discussion that follows and also to the accompanying drawings, illustrative approaches to the disclosed devices and methods are shown in detail. Although the drawing figures represent some possible approaches, the drawing figures are not necessarily to scale and certain features may be exaggerated, removed, or partially sectioned to better illustrate and explain the present disclosure. Further the descriptions set forth herein are not intended to be exhaustive or otherwise limit or restrict the claims to the precise forms and configurations shown in the drawings and disclosed in the following detailed description.

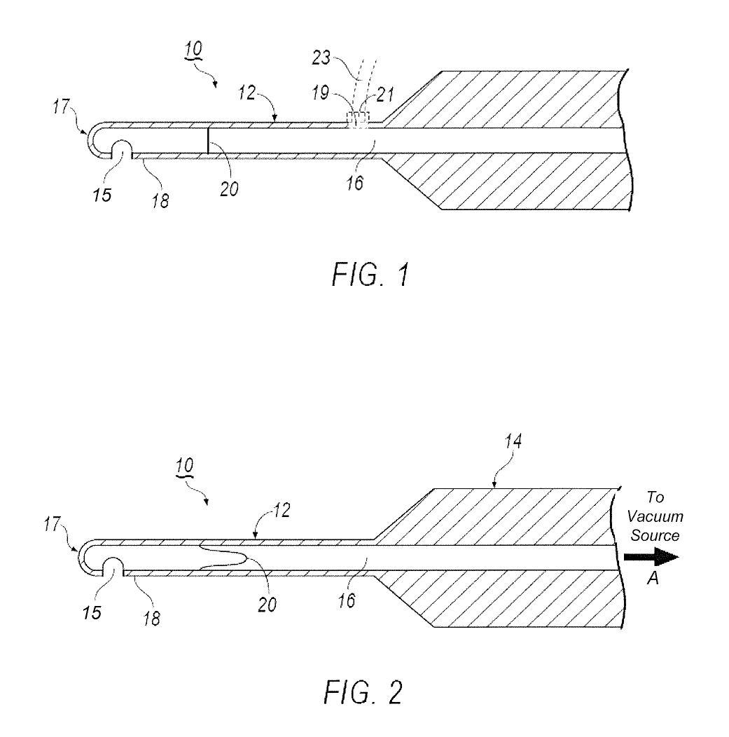

FIG. 1 illustrates a partial cross-sectional view of a capsulorhexis device 10. In one exemplary configuration, capsulorhexis device 10 is a two-piece design that includes a tip portion 12 that is attached to a handle member 14. However, it is understood that capsulorhexis device 10 may be configured to be a single piece design.

In one exemplary arrangement a vacuum channel 16 is formed such that it extends through both handle member 14 and tip portion 12. A port 15 is formed adjacent a distal end 17 of tip portion 12. More specifically, port 15 is disposed through a side wall 18 of tip portion 12 and is in communication with vacuum channel 16. A flexible membrane 20 is disposed within vacuum channel 16, spaced proximally from port 15. A vacuum source (not shown) is operatively connected with vacuum channel 16.

In an alternative construction (illustrated in phantom in FIG. 1), tip portion 12 is configured with a proximal vacuum port 19 that is disposed adjacent to a proximal end of tip portion 12. Proximal vacuum port 19 is configured to receive a fitting member 21 that is operatively connected to a vacuum supply via suitable tubing.

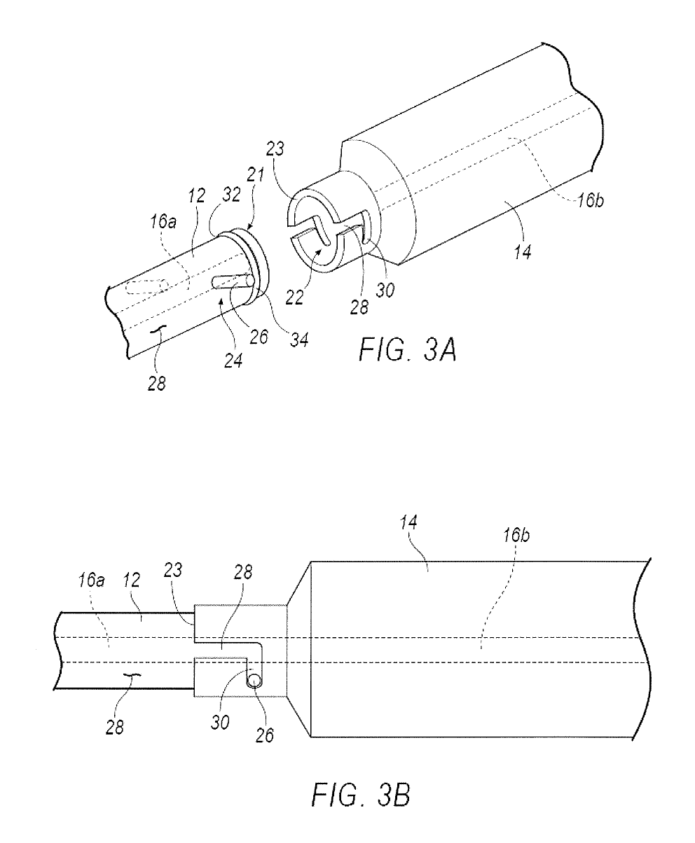

In one exemplary arrangement, tip portion 12 is integrally attached to handle member 14. In another exemplary configuration, tip portion 12 may be selectively removable from handle member 14. For example, as shown in FIGS. 3A and 3B, a proximal end 21 of tip portion 12 may be sized to be received within a chamber 22 formed in distal end 23 of handle member 14. An attachment mechanism 24 may be used to selectively secure tip portion 12 to handle member 14.

In one exemplary arrangement, attachment mechanism 24 is configured as a bayonet style attachment mechanism. More specifically, retaining arms 26 are disposed on an outside surface 28 of tip portion 12, adjacent proximal end 21. Distal end 22 of handle member 14 comprises retaining channels 28 that are in communication with a retaining groove 30. A groove 32 is formed on outside surface 28 of tip portion 12, proximally of retaining arms 26. A seal member 34 is disposed within groove 32.

With seal member 34 disposed within groove 32, proximal end 21 of tip portion 12 is positioned within chamber 22 of distal end 23 of handle member 14. As proximal end 21 is inserted within chamber 22, with each retaining arm 26 being positioned in a corresponding retaining channel 28. Tip portion 12 is then twisted to secure retaining arms 26 within retaining groove 30, thereby locking tip portion 12 to handle member 14. Seal member 34 becomes compressed within chamber 21, thereby creating a fluid-type seal between tip portion 12 and handle member 14. Once connected, vacuum channel 16a of tip portion 12 then aligns with and mates with vacuum channel 16b of handle member 14 to create a single vacuum channel 16.

While a bayonet style attachment mechanism has been described, it is understood that other selectively releasable attachment mechanisms 24 may be utilized. For example, a threaded attachment may also be used.

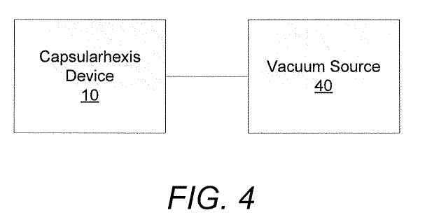

Capsulorhexis device 10, is operatively connected to a vacuum source (e.g., see vacuum source 40 in FIG. 4). Vacuum (represented by arrow A in FIG. 2) is introduced through vacuum channel 16. In one exemplary arrangement, the introduction of vacuum is controlled by a foot pedal that permits vacuum to be selectively turned on or off. The vacuum is built to a predetermined level at port 15 due to membrane 20. For example, the predetermined level of vacuum may be approximately in a range of 0 to 700 mmHg (e.g., 300 mmHg). Other predetermined vacuum levels are also contemplated. More specifically, as shown in FIG. 2, membrane 20 is constructed of a suitable material, such as, for example, silicone, that permits a predetermined amount of vacuum to pass through membrane 20 and reach port 15. However, membrane 20 also serves to limit the degree of vacuum such that aspiration through vacuum channel 16 is prevented.

At the start of a capsulorhexis procedure, an initial incision is made in the cornea to introduce a cystotome into the eye. A small incision is then made with the cystotome in the center of the lens so generate a radial leaflet of tissue to grasp. The physician grasps the leaflet by applying port 15 to the leaflet. The vacuum through vacuum channel 16 allows the physician to grab and hold the leaflet of the capsular bag, without aspirating the capsular bag or damaging adjacent structures. Indeed, the degree of vacuum is limited by membrane 20 so as to provide a sufficient holding force, allowing the physician to tear in a circular motion. Further the physician may selectively turn the vacuum on and off to quickly and easily to reposition capsulorhexis device 10, as desired by the physician. In one exemplary configuration, the vacuum source may be selectively turn on and off by a remote foot pedal. Alternatively, capsulorhexis device 10 may be provided with an actuation button to selectively turn on and off the vacuum.

Because the vacuum may easily be turned on and off, the capsulorhexis device 10 provides precision control in grasping and re-grasping the leaflet, but utilizing a simplified construction. Accordingly, as the construction may be simplified, capsulorhexis device 10 need not be constructed of expensive materials, typically required of precision devices. Indeed, capsulating device 10 need not be reusable, thereby minimizing the risk of bacteria. Capsulorhexis device 10 may be constructed to be entirely disposable, and be constructed of a variety of materials. For example, device 10 may be constructed of a suitable medical grade plastic or inexpensive material. For one exemplary embodiment, tip member 12 may be selectively detachable from handle portion 14 (such as, for example, the embodiment described in connection with FIGS. 3A-3B), such that tip member 12 may be disposable, with handle member 14 be constructed so as to be reusable.

Because the vacuum source provides the sufficient holding force, without requiring a forceps-type opening and closing action, the size of the initial incision in the cornea can be minimized. Indeed, in one exemplary arrangement, the initial incision is approximately 1 mm. With a smaller sized initial incision, trauma to the cornea can be minimized, thereby promoting quicker healing for the patient.

It will be appreciated that the devices and methods described herein have broad applications. The foregoing embodiments were chosen and described in order to illustrate principles of the methods and apparatuses as well as some practical applications. The preceding description enables others skilled in the art to utilize methods and apparatuses in various embodiments and with various modifications as are suited to the particular use contemplated. In accordance with the provisions of the patent statutes, the principles and modes of operation of this invention have been explained and illustrated in exemplary embodiments.

It is intended that the scope of the present methods and apparatuses be defined by the following claims. However, it must be understood that this invention may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future examples. Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the invention and that the method and apparatus within the scope of these claims and their equivalents be covered thereby. In sum, it should be understood that the invention is capable of modification and variation and is limited only by the following claims.

1. A capsulorhexis device, comprising:
a tip portion;
a vacuum channel extending through at least the tip portion, the vacuum channel configured to couple to a vacuum source;
wherein the tip portion further includes a port in communication with the vacuum channel; and
a flexible membrane positioned within the vacuum channel; the flexible membrane permitting a predetermined level of vacuum to reach the port without causing aspiration through the vacuum channel.

2. The capsulorhexis device of claim 1, wherein the port is positioned adjacent a distal end of the tip portion.

3. The capsulorhexis device of claim 1, further comprising a handle portion that is operatively connected to the tip portion.

4. The capsulorhexis device of claim 3, wherein the tip portion is integrally formed with the handle portion and the vacuum channel extends from the port to a proximal end of the handle member.

5. The capsulorhexis device of claim 1, wherein the device is constructed of a non-metallic material.

6. The capsulorhexis device of claim 1, wherein the predetermined level of vacuum is approximately in a range of 0 to 700 mmHg.

7. The capsulorhexis device of claim 3, wherein the tip portion is selectively detachable from the handle portion.

8. The capsulorhexis device of claim 7, wherein:
the tip portion includes a first vacuum channel segment extending from the port to a proximal end of the tip portion; and
the handle portion includes a second vacuum channel segment extending from a distal end of the handle portion to a proximal end of the handle portion;
wherein the first and second vacuum channels align to for a continuous vacuum channel between the port and the proximal end of the handle portion.

9. The capsulorhexis device of claim 8, further including a seal member disposed between the handle portion and the tip portion.

10. The capsulorhexis device of claim 7, further comprising an attachment mechanism to selectively secure the tip portion to the handle portion.

11. The capsulorhexis device of claim 10, wherein the attachment mechanism is a bayonet style configuration.

12. The capsulorhexis device of claim 11, further comprising at least one retaining arm formed on an outside surface of the tip portion and at least one corresponding retaining channel formed in a distal end of the handle portion that is in communication with a retaining groove.

13. The capsulorhexis device of claim 12, wherein the tip portion further comprises a seal member disposed in a groove formed adjacent to a proximal end of the tip portion.

14. The capsulorhexis device of claim 7, wherein the tip portion is constructed of a non-metallic material.

15. (canceled)

16. The capsulorhexis device of claim 1, wherein the vacuum may be selectively communicated to the port.

17. The capsulorhexis device of claim 1, wherein the flexible membrane is secured to an interior wall of the vacuum channel, proximally of the port.

18. A disposable capsulorhexis device, comprising:
a handle portion;
a tip portion;
a vacuum channel extending through the tip portion and the handle portion, the vacuum channel configured to be coupled to a vacuum source;
wherein the vacuum may be selectively communicated to the port;
wherein the tip portion further includes a port positioned adjacent a distal end of the tip portion, the port being in communication with the vacuum channel; and
a flexible membrane secured to an interior wall of the vacuum channel, proximally of the port, the flexible membrane permitting a predetermined level of vacuum to reach the port without causing aspiration through the vacuum channel.

19. The capsulorhexis device of claim 18, wherein the tip portion is integrally formed with the handle portion and the vacuum channel extends from the port to a proximal end of the handle member.

20. The capsulorhexis device of claim 18, wherein the tip portion is selectively detachable from the handle portion.

21. The capsulorhexis device of claim 1, wherein the flexible membrane extends across the vacuum channel to stop aspiration of liquid through the vacuum channel while allowing a predetermined level of vacuum to reach the port.
